(19) United States
(12) Patent Application Publication
(10) Pub. No.: US 2024/0008759 A1
(43) Pub. Date: Jan. 11, 2024

(54) METHOD AND SYSTEM FOR MONITORING CARDIOPULMONARY FUNCTION USING ELECTRICAL IMPEDANCE TOMOGRAPHY

(71) Applicant: BILAB CO., LTD., Seongnam-si, Gyeonggi-do (KR)

(72) Inventors: Hun WI, Seongnam-si, Gyeonggi-do (KR); Jun CHANG, Seoul (KR); Geuk Young JANG, Yongin-si, Gyeonggi-do (KR)

(21) Appl. No.: 17/768,467

(22) PCT Filed: Aug. 13, 2020
(86) PCT No.: PCT/KR2020/010784

§ 371 Date: Apr. 12, 2022

(30) Foreign Application Priority Data
Oct. 16, 2019 (KR) 10-2019-0128583

(51) Int. Cl. A61B 5/0536; A61B 5/0205; A61B 5/00; G06T 7/00

(52) U.S. Cl. CPC A61B 5/0536; A61B 5/0205; A61B 5/743; A61B 5/0037; A61B 5/7264; G06T 7/0012; A61B 5/7271; A61B 2505/03

(57) ABSTRACT

The present inventive concept relates to a method and device for monitoring cardiopulmonary function using electrical impedance tomography, and more specifically, to a method and system for monitoring cardiopulmonary function using electrical impedance tomography, whereby lung collapse and hyperinflation are monitored in real-time during a mechanical ventilation treatment process by using a single monitoring device, and information on a plurality of hemodynamic diagnostic parameters changing in real-time during the mechanical ventilation treatment process can be provided. According to the present inventive concept, it is possible to take selectively electrical impedance tomography of blood vessels at any part of a body such as the chest, neck, arms, legs, etc., and to monitor hemodynamic diagnostic parameters including a stroke volume, a cardiac output, a peripheral vascular resistance, etc. In addition, according to the present inventive concept, it is possible to monitor in real-time state parameters for each region of lungs including lung compliance data, ventilation delay data, etc., by using the same monitoring device.

### TECHNICAL FIELD

The present inventive concept relates to a method and apparatus for monitoring cardiopulmonary function using electrical impedance tomography, and more specifically, to a method and system for monitoring cardiopulmonary function using electrical impedance tomography, which performs real-time monitoring collapse and hyperinflation of lungs in the course of mechanical ventilation treatment process by using a single monitoring device, and provides information on a plurality of hemodynamic diagnostic parameters that can be changed in real-time during a mechanical ventilation treatment process.

### BACKGROUND ART

What is described in this section is merely for providing background information for embodiments of the present inventive concept and thus does not constitute prior arts.

Transpulmonary Thermodilution (TPTD) and arterial blood pressure Pulse Contour Analysis (PCA) are currently used to observe hemodynamic diagnostic parameters during the treatment process for critically ill patients. TPTD and arterial blood pressure PCA methods are invasive methods for observing a central vein and artery by inserting a catheter into the central vein and artery of a subject.

TPTD is a method of measuring blood flow quantity by injecting a temperature indicator to a subject and measuring temperature change, in which there is a problem, in view that it takes more than 1 minute for one time measurement and the number of repeated measurements is limited.

Arterial blood pressure PCA is a method of calculating hemodynamic diagnostic parameters by measuring arterial blood pressure waveforms including maximal blood pressure (systolic blood pressure) and minimal blood pressure (diastolic blood pressure), and then predicting peripheral vascular resistance. At this time, an error can be occurred due to predicting the peripheral vascular resistance. There is also a problem in light that arterial blood pressure PCA takes about 20 seconds for one time measurement.

There is a non-invasive hemodynamic monitoring method that measures bioimpedance or bioreactance signals with attaching a plurality of electrodes to a chest, as a non-invasive method which is different from the above methods, for observing hemodynamic diagnostic parameters in critically ill patients.

Korea Patent Publication No. 10-2014-0058570 (Invention title: method and system for monitoring hemodynamics) discloses a system for monitoring the hemodynamics of a subject, comprising a signal generating system configured to provide at least one output electrical signal and deliver the output signal to an organ of the subject; a demodulation system configured to receive an input electrical signal sensed from the organ in response to the output electrical signal and to modulate the input signal by using the output signal so as to provide respective in-phase and quadrature-phase components of the input signal; and a processing system configured to monitor hemodynamics based on the in-phase and quadrature-phase components.

While the Korea Patent Publication No. 10-2014-0058570 is a method for monitoring non-invasive hemodynamics, there is a problem that the measurement signal is affected by various causes such as respiration, movement of internal organs, and movement of the subject as well as a blood flow of the heart. That is, there is a problem in that it is difficult for the Korea Patent Publication No. 10-2014-0058570 to extract only the blood flow component from the measurement signal. Therefore, there is a need for a device of monitoring hemodynamics diagnostic parameters, which can perform real-time monitoring with non-invasive and accurate detection values during the treatment process of critically ill patients.

In addition, there are many cases in which a mechanical ventilation system is used in the treatment process of critically ill patients. As an example, in mechanical ventilation using a ventilator, positive end-expiratory pressure (PEEP) is provided to the subject through the airway for recovering a collapsed part of the lungs.

However, due to the large positive end-expiratory pressure value during the end-expiratory pressure treatment process, an overdistension area is generated in the lungs of the subject so that it can cause acute lung injury. This situation causes a problem that worsens the condition of the critically ill patients or, in severe cases, leads them to death. That is, an unoptimized setting in mechanical ventilation for pulmonary respiration can adversely affect the prognosis of the patient as well as the occurrence of complications.

Up to now, pulmonary ventilation control has relied heavily on physiological parameters that reflect overall lung function. And complications of lung disease often occur when treatment is performed depending only on the overall information of the lungs. Therefore, there needs a pulmonary protective ventilation protocol that acknowledges the information on the local ventilation distribution for each area of the lungs and sets the most suitable ventilation to the patient.

Up to now, the information commonly utilized in clinical practice is through CT, MRI, and chest X-ray. However, the imaging in these imaging methods is performed to check the conditions of the patient before or after treatment. That is, since it is impossible to perform real-time monitoring for the conditions of the patient along with the mechanical ventilation treatment process, there are limitations in providing customized treatment to patients after immediately checking how each area of the lungs responds during the treatment process.

The method of utilizing electrical impedance tomography makes image for the distribution of air inside the lungs while increasing or decreasing PEEP during mechanical ventilation and distinguishes from collapsed and hyperinflated regions by analyzing the image. In addition, an appropriate PEEP value to treat collapse while minimizing hyperinflation is also presented.

This electrical impedance tomography method requires the processes of measuring EIT data during increase and decrease of PEEP over several minutes and performing image restoration and image data analysis after the measurement process is finished. Therefore, there needs a method for monitoring in real-time how the collapse and hyperinflation areas change when the medical staff makes the PEEP changed.

Therefore, there needs a monitoring device capable of monitoring in real-time the conditions such as collapse, hyperinflation, tidal volume, and hemodynamic diagnostic parameters of the lungs during the treatment of critically ill patients.

### DISCLOSURE

### Technical Problem

Accordingly, it is an objective of the present inventive concept to provide a method and system for monitoring cardiopulmonary function using electrical impedance tomography, which is capable of monitoring lung collapse and hyperinflation in real-time in the course of mechanical ventilation treatment process.

It is another objective of the present inventive concept to provide a method and system for monitoring cardiopulmonary function using electrical impedance tomography, which can provide information on a plurality of hemodynamic diagnostic parameters that are changed in real-time during a mechanical ventilation treatment process.

It is another objective of the present inventive concept to provide a method and system for monitoring cardiopulmonary function using electrical impedance tomography, which can monitor in real-time both lung collapse and hyperinflation by using a single monitoring device in the course of mechanical ventilation treatment process and can provide information on a plurality of hemodynamic diagnostic parameters that are changed in real-time during a mechanical ventilation treatment process.

It is another objective of the present inventive concept to provide a method and system for monitoring cardiopulmonary function using electrical impedance tomography, which can selectively take electrical impedance tomography of blood vessels in any part of the chest, neck, arms, legs, etc., and is capable of monitoring hemodynamic diagnostic parameters.

It is another objective of the present inventive concept to provide a method and system for monitoring cardiopulmonary function using electrical impedance tomography, which can provide in real-time the states of lung collapse, hyperinflation, tidal volume, etc. as images and numerical values according to PEEP control and can support that medical staffs find the most appropriate PEEP for a patient.

It is another objective of the present inventive concept to provide a method and system for monitoring cardiopulmonary function using electrical impedance tomography, which can identify the state of each area of the lung in real-time through images and numerical values and can prevent in advance the problems such as lung collapse and hyperinflation.

### Technical Solution

In order to solve the above technical problems, a system for monitoring cardiopulmonary function using electrical impedance tomography according to an embodiment of the present inventive concept comprises: an electrode unit configured to measure impedance data by attaching a plurality of electrodes to at least one region with blood vessels of chests, a neck, arms, legs, or wrists of a subject; an image restoring unit configured to restore an EIT image by extracting blood flow impedance data from the measured impedance data; and an EIT control module configured to set a region of interest in the restored EIT image, extract a blood flow change signal based on change amount of pixel values inside the region of interest, and calculate hemodynamic diagnostic parameters by using the extracted blood flow change signal.

Wherein, the EIT control module is preferably configured to calculate a stroke volume by using the extracted blood flow change signal.

Wherein, the EIT control module is preferably configured to calculate a cardiac output by computing (multiplying) a heart rate measured from the subject on the calculated stroke volume.

Wherein, the EIT control module is preferably configured to calculate a peripheral vascular resistance by computing (dividing) the blood pressure measured from the subject on the cardiac output.

Wherein, the EIT control module is preferably further configured to calculate lung perfusion by extracting blood flow change in the lung region of the subject.

Wherein, the EIT control module is characterized to be preferably further configured to preset weights according to gender, age, height, and weight of the subject, and apply the preset weights to the stroke volume calculation.

The system is characterized to further comprise a display unit configured to display an EIT image restoring a blood flow change signal over time generated based on a signal detected in real-time through the electrode unit, a graph of hemodynamic diagnostic parameters proportional to the EIT image, and numerical values.

In order to solve the above technical problems, a system for monitoring cardiopulmonary function using electrical impedance tomography according to an embodiment of the present inventive concept comprises: an electrode unit configured to measure impedance data by attaching a plurality of electrodes to a chest of a subject for monitoring lung collapse and hyperinflation in real-time during the mechanical ventilation treatment process; a sensing unit configured to measure the pressure data of air applied to the subject in the mechanical ventilation treatment process; an image restoring unit configured to restore an EIT image by extracting airflow impedance data from the measured impedance data; and an EIT control module configured to obtain a plurality of airflow EIT images in order to extract an airflow change signal from the restored EIT image, extract the airflow change signal in each pixel based on the change in the pixel value from the obtained EIT image, and calculate respiratory dynamics diagnostic parameters by using the extracted airflow change signal.

Wherein, the EIT control module is characterized to preferably further configured to calculate a stroke volume by using the extracted airflow change signal.

Wherein, the EIT control module is characterized to be preferably further configured to calculate the lung compliance data in each pixel by computing a ventilation volume and air pressure data extracted from each pixel, and the system further comprises a display unit configured to display the lung compliance data as images, wherein the lung compliance data is changed in synchronization with time change.

Wherein, the EIT control module is characterized to be preferably further configured to calculate a ventilation delay data by computing time taken to reach a volume corresponding to 40% of maximum volume from start of inspiration in the corresponding pixel with respect to the time taken from the start of inspiration to the end of inspiration, and wherein the display unit is characterized to be preferably further configured to display the ventilation delay data as images, wherein the ventilation delay data is changed in synchronization with time change.

Wherein, the EIT control module is characterized to be preferably further configured to determine an area as collapse and hyperinflation of the lungs, by combining results determined from lung compliance data and the ventilation delay data, wherein the area in which the lung compliance data is reduced within each cycle of respiration is determined as the collapse area and the hyperinflation area of the lungs, and the area in which the ventilation delay data increases within each cycle of respiration is determined as the collapse area of the lungs.

Wherein, the EIT control module is characterized to be preferably further configured to calculate results of lung compliance data and ventilation delay data according to changes where positive end-expiratory pressure (PEEP) increases and decreases, and the display unit is characterized to be preferably further configured to display the collapsed and overinflated areas of the lungs that are changed in synchronization with the change of the positive end-expiratory pressure.

In order to solve the above technical problems, a method for monitoring cardiopulmonary function using electrical impedance tomography according to an embodiment of the present inventive concept comprises: measuring impedance data by attaching a plurality of electrodes to chest of a subject; measuring air pressure data and air volume data applied to the subject during the mechanical ventilation treatment process; restoring a blood flow EIT image and an airflow EIT image by extracting a blood flow impedance data and an airflow impedance data from the measured impedance data; extracting a blood flow change signal based on a change amount of a pixel value in a region of interest, by obtaining a plurality of EIT images for a predetermined time, and setting the a blood vessel regions (or vascular region) as the region of interest in the obtained EIT image, in order to extract a blood flow change signal from the restored blood flow EIT image; extracting an airflow change signal in each pixel based on a change in pixel value from the obtained EIT image by obtaining a plurality of airflow EIT images for a predetermined time, in order to extract an airflow change signal from the restored airflow EIT image; and calculating hemodynamic diagnostic parameters by using the extracted blood flow change signal and respiratory dynamics diagnostic parameters by calculating the airflow change signal and air pressure data extracted from each pixel.

Wherein, the extracting of the blood flow change signal is characterized to preferably comprise extracting the blood flow change signal from the blood flow impedance data obtained from a body part with a blood vessel of a chest, neck, arms, legs, and wrists of the subject.

The method is characterized to preferably comprise displaying an EIT image obtained by reconstructing a blood flow change signal generated based on a signal detected in real-time from the electrode and displaying a graph and a numerical value of a hemodynamic diagnostic variable calculated from the EIT image.

The extracting of the airflow change signal is characterized to preferably comprise extracting an airflow change signal including tidal volume, pulmonary compliance data, or ventilation delay data by using an airflow impedance data and air pressure data obtained from a region of a neck and chest of the subject, where there is a flow of air due to respiration.

The method is characterized to further comprise displaying an EIT image restoring a blood flow change signal generated based on a signal detected in real-time from the electrodes, a graph of hemodynamic diagnostic parameters calculated from the EIT image, and a numerical value.

### Advantageous Effects

A method and system for monitoring cardiopulmonary function using electrical impedance tomography according to the present inventive concept enables a subject to be monitored in real-time using electrical impedance tomography. That is, since the present inventive concept does not cause unnecessary pain and does not require a special treatment process to the subject in the process for confirming conditions of the subject, it is possible to promote convenience in use and safely monitor the subject.

In addition, it is effective for the present inventive concept to confirm the information of a plurality of hemodynamic diagnostic parameters that are changed in real-time from the subject during the mechanical ventilation treatment process.

And it is possible for the present inventive concept to monitor collapse and hyperinflation of the lungs in real-time in the course of mechanical ventilation treatment using a single monitoring device and provide information on a plurality of hemodynamic diagnostic parameters that are changed in real-time during the mechanical ventilation treatment process.

Therefore, the present inventive concept is effective in economic and space points of views, because the system according to the present inventive concept can check various diagnostic parameters through a single monitoring device in a case that it is difficult to combine a number of machines due to space constraints such as an intensive care unit.

In addition, it is possible for the present inventive concept to selectively perform electrical impedance tomography to blood vessels in any part of the chest, neck, arms, legs, wrists, etc., and monitor hemodynamic diagnostic parameters. Therefore, the present inventive concept takes advantageous of being very efficiently used in a medical environment, because electrical impedance tomography can be performed on other parts of the body with blood vessels, even in the case of critically ill patients who have difficulty in installing electrodes in the chest region.

And it is possible for the present inventive concept to provide pulmonary compliance data and ventilation delay data in real-time as images and numerical values for health care providers to decide the states of collapse and/or hyperinflation of the lungs according to PEEP control, and it is possible to assist health care providers in finding the most appropriate PEEP for their patients. Therefore, the present inventive concept makes the health care providers confirm the state of each area of the lungs in real-time through images and numerical values and makes them plan to prevent problems such as collapse and hyperinflation of the lungs in advance.

### DESCRIPTION OF DRAWINGS

FIG. 1 shows a display as an example of a system for monitoring cardiopulmonary function using electrical impedance tomography according to an embodiment of the present inventive concept.

FIG. 2a to FIG. 2d show blood flow images and ventilation images being changed in restored time sequence based on the impedance image detected from the electrode unit attached to the chest.

FIG. 3 shows an exemplary diagram in which electrodes are attached to body parts capable of scanning an EIT image for monitoring cardiopulmonary function in a monitoring system 100 according to an embodiment of the present inventive concept.

FIG. 4a to FIG. 4d show exemplary diagrams in which electrodes are attached to the chest region for monitoring cardiopulmonary function.

FIG. 5 shows a diagram illustrating a state of use in which electrodes are attached to a wrist region.

FIG. 6a to FIG. 6c show blood flow EIT images scanned from the wrist, stroke volume graph (blue), and electrocardiogram graph (red) in time sequence.

FIG. 7a and FIG. 7b show diagrams illustrating states of use in which electrodes are attached around a neck.

FIG. 8a to FIG. 8c show blood flow EIT images scanned from the neck, stroke volume graph, and electrocardiogram graph in time sequence.

FIG. 8d shows a result where the pattern extraction unit 112 extracts a pattern data corresponding to a specific component corresponding to the upper airway signal and performs filtering the pattern data.

FIG. 9 shows a view showing an overall control configuration of the system for monitoring cardiopulmonary function using electrical impedance tomography according to an embodiment of the present inventive concept.

FIG. 10a shows a diagram illustrating a configuration for reconstructing extracted pattern data according to an embodiment of the present inventive concept.

FIG. 10b shows frequency patterns of mixed signal 401, PCA pattern data 402, and ICA pattern data 403.

FIG. 10c shows that EIT data 500 corresponding to a composite signal is generated by reconstruction.

FIG. 11 shows a graph showing the sum of pixel values based on a blood flow change signal in a region of interest (ROI) set in the heart and lungs.

FIG. 12a shows an EIT image according to cardiopulmonary function detected during an animal experiment and shows a reconstructed respiration image that varies according to a respiration volume.

FIG. 12b shows a process of generating a tidal volume graph by extracting a respiration change signal from a change in pixel values in a respiration EIT image.

FIG. 13a shows an image of pulmonary compliance obtained from an animal undergoing mechanical ventilation through an animal experiment.

FIG. 13b shows ventilation delay images obtained through an animal experiment.

FIG. 14a shows change states of respiration EIT images that are changed according to an increase of PEEP for a specific pixel.

FIG. 14b shows an A/P ratio value graph showing the amount of change in the ventilation EIT image.

FIG. 15 shows exemplary diagrams showing in real-time state parameters for each area of the lungs in the monitoring system of the present inventive concept.

### MODES FOR THE INVENTIVE CONCEPT

Hereinafter, the embodiments disclosed in the present specification will be described in detail with reference to accompanying drawings, but the same reference numbers are assigned to the same or similar components regardless of reference numerals of the drawings, and redundant description thereof will be omitted. Suffixes “part” and “unit”, “module” and “part”, “unit” and “part”, “device” and “system”, “critically ill patient” and “patient” and “subject”, “blood flow change signal”, and “blood flow change information” etc. for components used in the description below are given or mixed in consideration of only the ease of writing the specification, and do not have distinct meanings or roles by themselves.

In addition, in describing the embodiments disclosed in the present specification, if it is determined that detailed descriptions of related known technologies may obscure the gist of the embodiments disclosed in this specification, the detailed description thereof will be omitted.

In addition, the accompanying drawings are only for easy understanding of the embodiments disclosed in the present specification, and the technical idea disclosed in the present specification is not limited by the accompanying drawings, and should be understood to include all modifications, equivalents and substitutes included in the idea and scope of the present inventive concept.

Terms including an ordinal number, such as first, second, etc., may be used to describe various components, but the components are not limited by the terms. The above terms are used only for the purpose of distinguishing one component from another.

When a component is referred to as being “connected” or “accessed” to another component, it may be directly connected to or accessed to the other component, but it should be understood that other components may exist in between. On the other hand, when it is mentioned that a certain component is “directly connected” or “directly accessed” to another component, it should be understood that no other component is present in the middle.

The singular expression includes the plural expression unless the context clearly dictates otherwise.

In the present application, terms such as “comprises” or “have” are intended to designate that a feature, number, step, operation, component, part, or combinations thereof described in the specification exists, but it should be understood that this does not preclude the possibility of addition or existence of one or more other features or numbers, steps, operations, components, parts, or combinations thereof.

Hereinafter, embodiments of the present inventive concept will be described in detail with reference to the drawings. It is apparent to those skilled in the art that the present inventive concept may be embodied to other specific forms in the range without departing from the spirit and essential features of the present inventive concept.

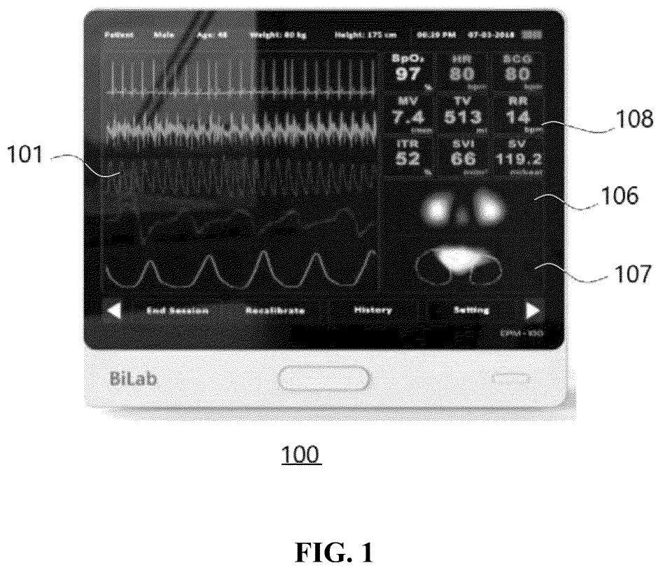

FIG. 1 shows an exemplary diagram of a display for a cardiopulmonary function monitoring system using electrical impedance tomography according to an embodiment of the present inventive concept.

A cardiopulmonary function monitoring system (hereinafter referred to as a “monitoring system”) using electrical impedance tomography according to an embodiment of the present inventive concept is non-invasively measures a change in blood flow over time and displays the change. In particular, the monitoring system of the present inventive concept takes EIT (Electrical Impedance Tomography) images from various parts of the body where blood vessels are located, and extracts information on changes in blood flow over time from the scanned EIT images. And it is characterized in that the monitoring system calculates hemodynamic diagnostic parameters including a stroke volume, a cardiac output, a peripheral vascular resistance, etc. by using the information and displays these parameters as images or Arabic numerals and letters.

The monitoring system 100 according to an embodiment of the present inventive concept can display oxygen saturation (SpO₂) data, heart rate (HR) data, Seismocardiogram (SCG) data, and minute ventilation (MV) data, tidal volume (TV) data, respiratory rate (RR) data, end-expiratory lung volume (EELV) data, inspiratory expiratory ratio (I:E ratio), stroke volume index (SVI), stroke volume (SV) data measured from a subject with letters and Arabic numerals 108.

The monitoring system 100 can display states related to the real-time measured heart rate, stroke volume, pulmonary ventilation, pulmonary perfusion respiration, and movement of blood flow as graph waveforms 101. In addition, the monitoring apparatus 100 can display the pulmonary ventilation impedance image 106 that is changed according to respiration, the pulmonary perfusion impedance image 107 that are changed according to blood flow, and the blood flow impedance image in real-time.

All data displayed on the monitoring system 100 are values based on signals sensed from the measurement target parts of the subject, and the monitoring system can display them in various ways by using numerical values, waveforms, images, and various colors.

The pulmonary ventilation impedance image 106 and the pulmonary perfusion impedance image 107 are restored from the pulmonary ventilation impedance data and the pulmonary perfusion impedance data received from the EIT device. As shown in FIG. 1, the pulmonary ventilation impedance image and the pulmonary perfusion impedance image are those imaging the inside of the chest of the subject and specific areas for the sensed values are displayed in different colors.

The pulmonary ventilation impedance data is a data obtained during the pulmonary ventilation process of the subject, the pulmonary ventilation process may be a process of moving air in and out of the lungs in a process in which the subject continuously and periodically breathes air.

Pulmonary perfusion impedance data is a data that can know the degree of blood inside the lungs of the subject and can confirm how evenly the blood is located in both lungs of the subject.

Accordingly, the pulmonary perfusion impedance data can be used for observing and diagnosing pulmonary vascular diseases of pulmonary embolism, thrombosis, tumor, lung cancer, tuberculosis, and granulomas; obstructive diseases of chronic bronchitis, emphysema, bronchial asthma, and bronchiectasis; and other diseases of pneumonia, pulmonary infarction, pleural effusion, and pneumothorax.

And the blood flow impedance data is a data that can know the degree of changes due to the movement of blood flow in the heart and major blood vessels of the subject, and the blood flow impedance data can see the heart rate, blood flow movement speed, and oxygen respiration volume according to the blood flow movement speed, changes according to blood flow movement in the major blood vessels inside the chest.

As described above, the monitoring system 100 according to an embodiment of the present inventive concept can display various measurement signals based on the impedance data and biosignals of the subject measured in real-time. Therefore, in addition to the data as shown, it is possible to display more diverse data based on pathological condition of the subject and to variously combine the displayed position, number, size, etc.

As an example, the monitoring system 100 according to an embodiment of the present inventive concept can display blood pressure data, end-tidal carbon dioxide partial pressure data, temperature data, and the like. In addition, it is possible to display together bio-signals for hemodynamic changes inside the heart such as a heart ballistic system and heart vibration waves.

In addition, the monitoring system 100 according to an embodiment of the present inventive concept monitors collapse and hyperinflation of the lungs in real-time in the course of mechanical ventilation treatment process, can display images in real-time that change according to the time change depending on the collapse and hyperinflation of the lungs. This will be described later part of the specification.

That is, the monitoring system 100 according to an embodiment of the present inventive concept measures various data related to the aforementioned hemodynamic diagnostic parameters and display them, in addition, the data according to collapse and hyperinflation of the lungs can be displayed in real-time and will be described later.

The monitoring system 100 according to an embodiment of the present inventive concept can display the respiration for each region of the subject and its corresponding cardiac motion, blood flow change, etc. as images.

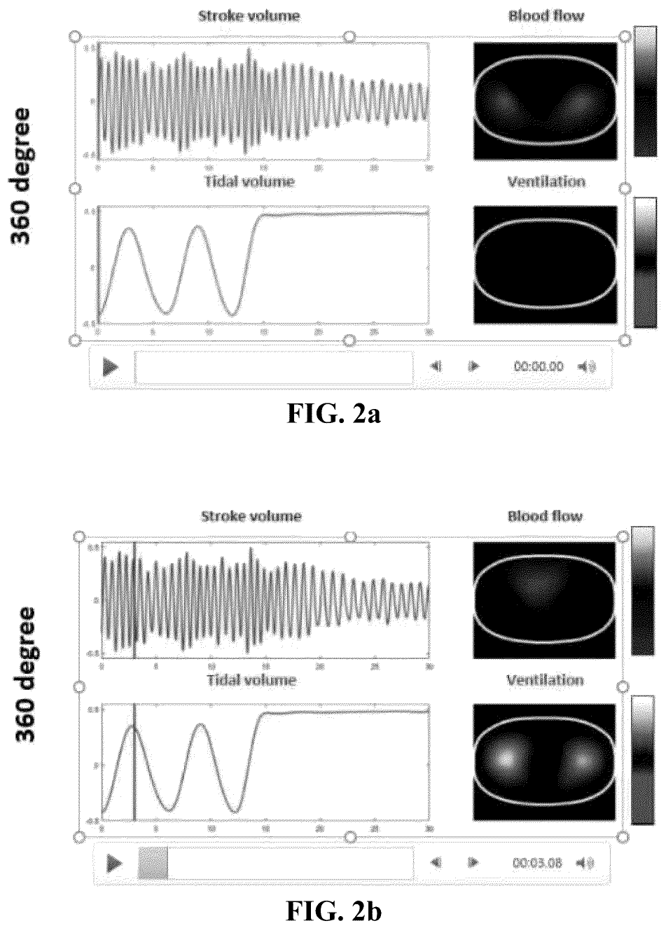

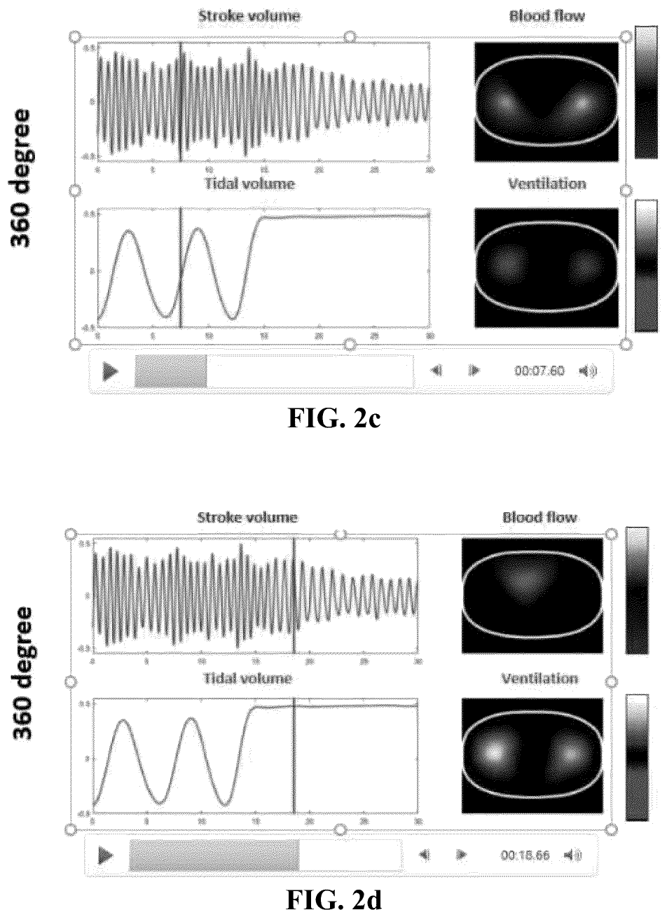

FIG. 2a to FIG. 2d show blood flow images and ventilation images that change in time sequence order restored based on the impedance image sensed from the electrode unit attached to the chest. In addition, it is possible to see the waveform according to the stroke volume and tidal volume moving in conjunction with the blood flow image and ventilation image. The blood flow image and ventilation image, and their resulting waveforms of the stroke volume and tidal volume can be measured and displayed in real-time.

The monitoring system 100 according to an embodiment of the present inventive concept obtains more than 100 frames of blood flow images per second made of the illustrated EIT imaging (can adjust to more than 25 frames per second when only airflow changes are imaged).

Then, the system sets a region of interest (ROI) in the obtained blood flow images and extracts a blood flow change signal from changes in pixel values in the region of interest. The stroke volume is calculated by using the extracted blood flow change signal.

Therefore, if the blood flow change signal is extracted from the change in the pixel values in the region of interest, the stroke volume can be calculated from the extracted blood flow change signal, and the cardiac output is calculated by computing the calculated stroke volume on the measured heart rate (HR). In addition, the peripheral vascular impedance is calculated by computing the cardiac output and the measured blood pressure. The hemodynamic diagnostic parameters calculated as above, are accurately calculated to a stroke volume, a cardiac output, and a peripheral vascular impedance, as it becomes possible to obtain a blood flow change signal from changes in pixel values in blood flow image restored from the scanned EIT image.

Such a configuration in which hemodynamic diagnostic parameters according to cardiopulmonary function can be confirmed in real-time is very useful for real-time observation of hemodynamic function recovery of a critically ill patient when fluid therapy is used for early recovery of the critically ill patient. Therefore, the monitoring system (i.e., apparatus or device) 100 of the present inventive concept can display real-time changing waveforms and images related to hemodynamic diagnostic parameters like a moving picture (i.e., a video). Therefore, the medical staff can perform real-time confirmation of the recovery of the hemodynamic function of the critically ill patient through the monitoring system device 100 and can seek to accurately make a necessary diagnosis and prescription.

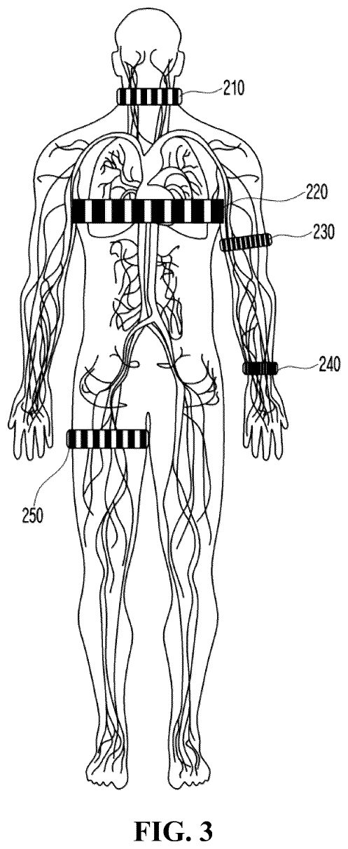

FIG. 3 shows an exemplary diagram in which electrodes are attached to a body part capable of scanning an EIT image for monitoring cardiopulmonary function in a monitoring system 100 according to an embodiment of the present inventive concept.

According to the present inventive concept, EIT images can be scanned at various parts of a human body where blood vessels are located. The carotid artery 210 around a neck, chest 220, arms 230, wrists 240, thigh 250, and the like can be found as representative parts in a human body where blood vessels are located. Therefore, it is possible to scan EIT images by attaching a plurality of electrodes to parts of the human body where blood vessels are located. In this case, in order to attach a plurality of electrodes to a circumference of each of body parts, a plurality of individual electrodes can be used, or a pad or a belt including a plurality of electrodes can be used.

That is, according to the present inventive concept, an image of blood flow can be obtained anywhere in the body where blood vessels are located. As an example, it may be difficult to attach electrodes to the chest of a critically ill patient. In this case, by attaching electrodes to other parts of the human body and scanning EIT images, information on changes of blood flow over time can be extracted from the scanned EIT images.

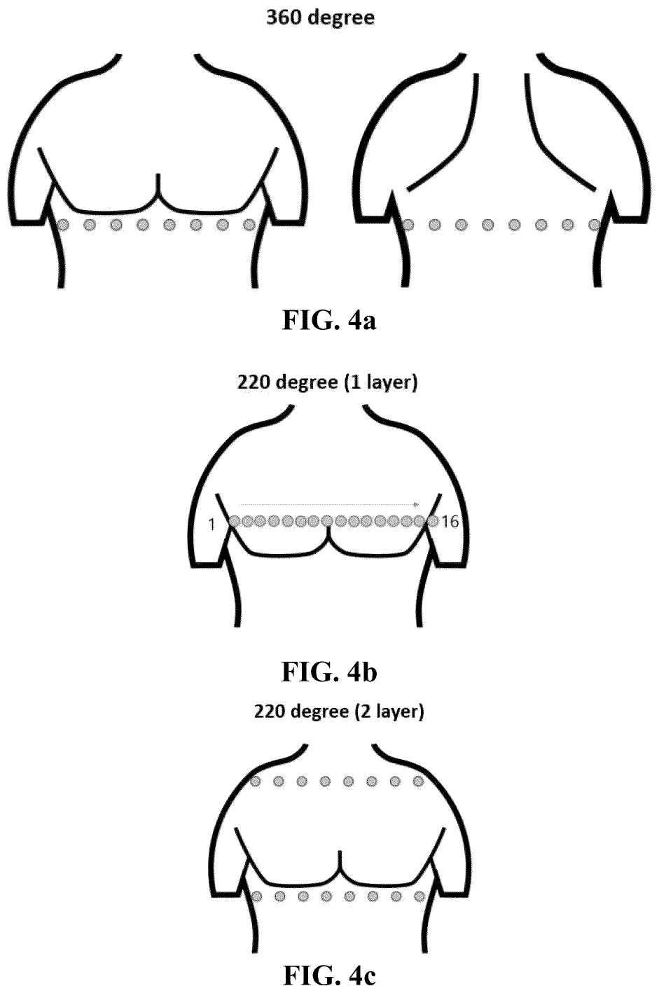

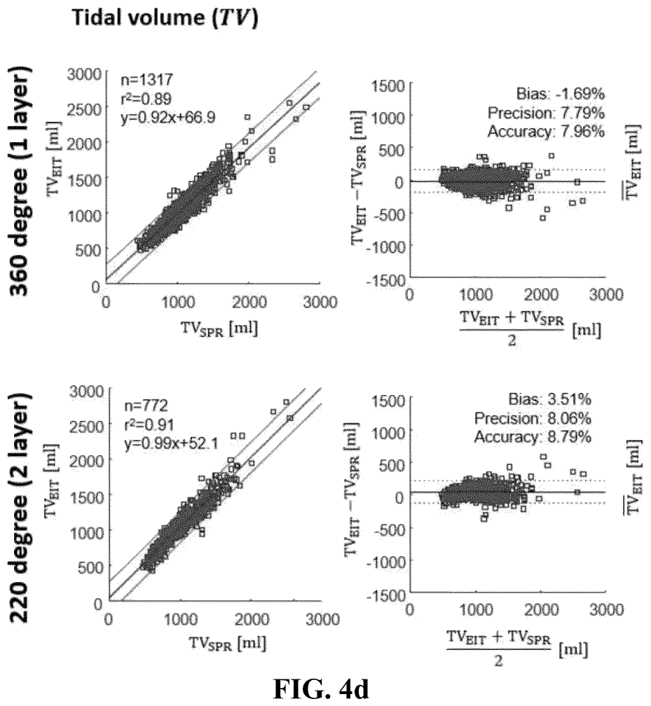

Next, FIG. 4 shows exemplary diagrams in which electrodes are attached to the chest area for monitoring cardiopulmonary function. FIG. 4a shows a case in which the electrodes are attached to the entire chest area at 360-degree. FIG. 4b shows a case in which the electrodes are attached to the chest area with a single layer at about 220-degree. FIG. 4c is a case in which electrodes are attached to the chest area with two layers at about 220-degree.

As described above, it is possible to obtain EIT images by attaching electrodes in various shapes to the chest area. FIG. 4d shows Tidal Volumes (TVs) which are compared with according to the electrodes attached to the chest area, wherein it can be seen that there is no significant difference in error range.

As in the case of the embodiment, when electrodes are attached to the chest area and EIT images are scanned, the scanned signals include a ventilation signal and a blood flow signal at the same time. In this case, a preprocessing operation to separate these two signal components is required, and after the components are separated, the ventilation and blood flow signals can be separately restored as images, respectively.

And when electrodes were attached to the entire chest area at 360-degree as shown in FIG. 4a, real-time restored images are sequentially shown in FIG. 2a to FIG. 2d according to time change.

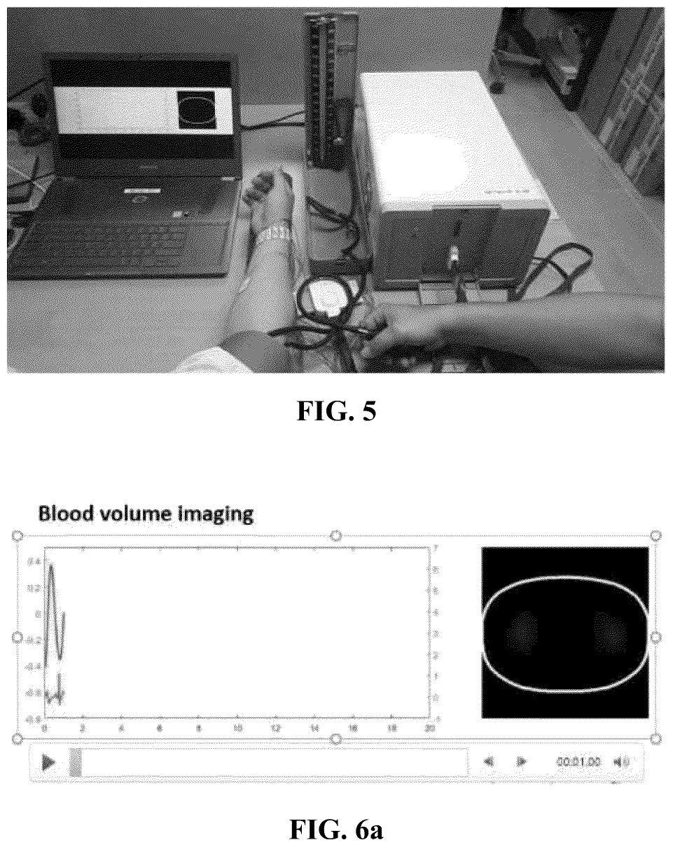

FIG. 5 shows a diagram illustrating a state of use in which electrodes are attached to a wrist area.

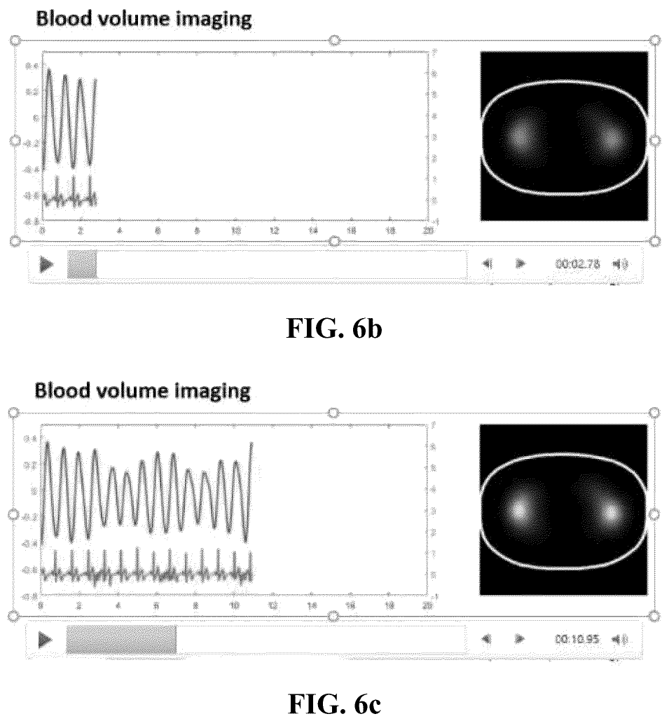

As in the case of an embodiment, when electrodes are attached to the wrist area and EIT image are scanned, blood flow images can be obtained from the blood flow impedance data sensed around the wrist. The blood flow EIT images, the stroke volume graph (blue color), and the electrocardiogram graph (red color) obtained in the above ways are shown in FIG. 6a to FIG. 6c in time sequence order. And, as shown in FIG. 6c, when the red-colored region in the blood flow EIT image is the most prominent, it can be seen that the blood flow is the most and the value of the stroke volume graph reaches the maximum.

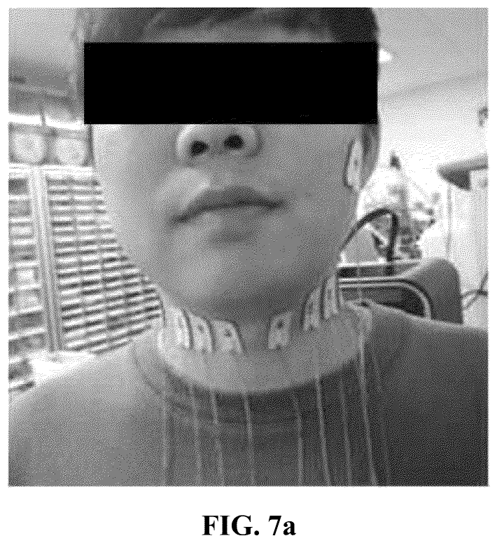

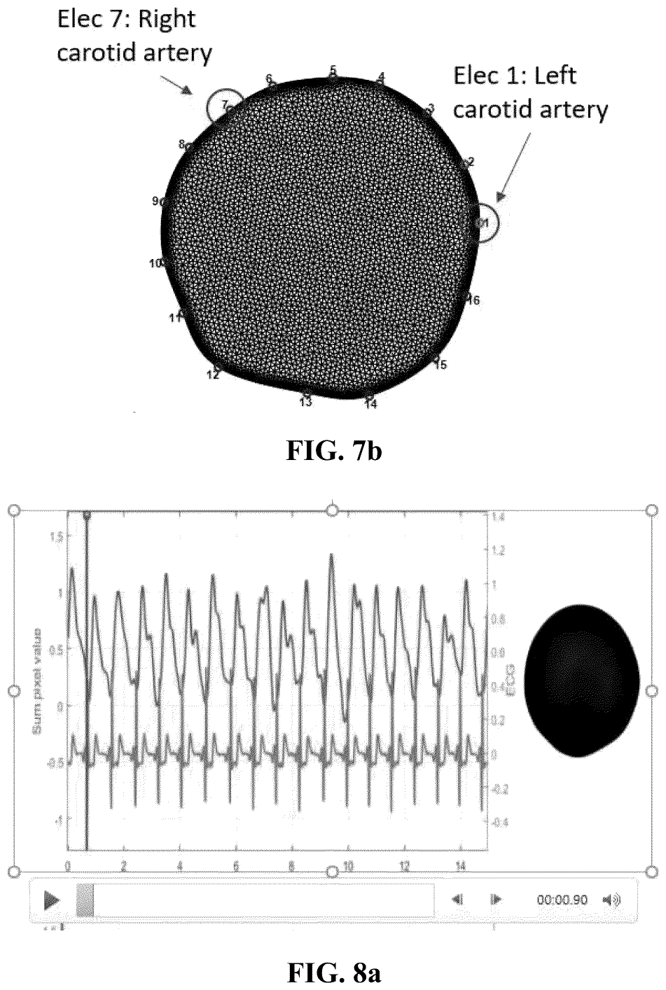

FIG. 7a and FIG. 7b show diagrams illustrating states of use in which electrodes are attached around a neck.

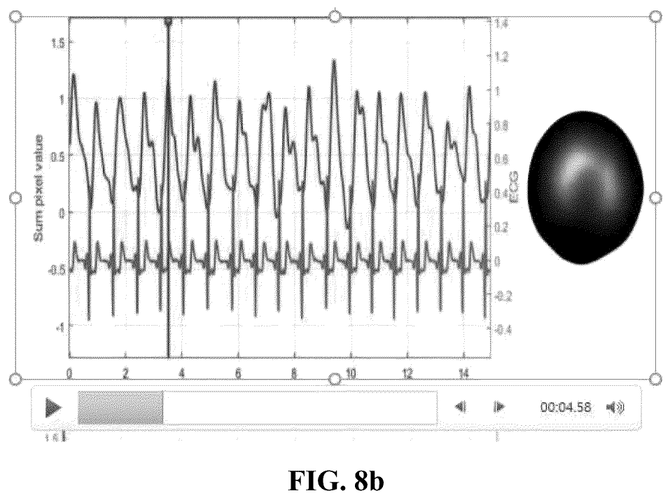

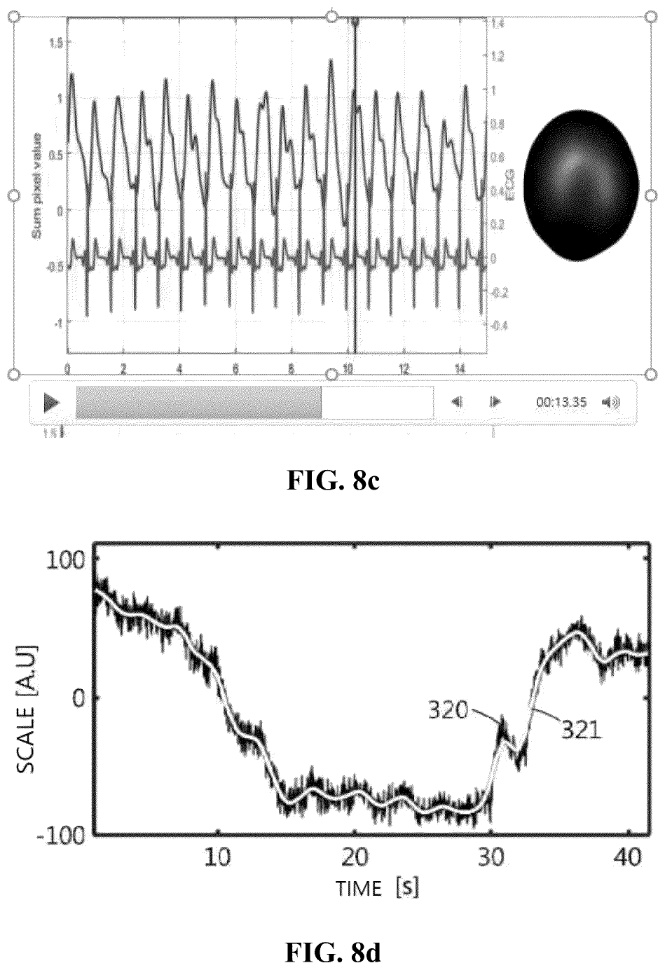

As in the case of an embodiment, when electrodes are attached around the neck and EIT images are scanned, blood flow images can be obtained from the blood flow impedance data sensed around the neck. The blood flow images obtained at this time are shown in FIG. 8a to FIG. 8c.

In an embodiment, only the components due to a specific physiological phenomenon are extracted from the composite signal affected by the change of electrical properties inside the human body according to a plurality of physiological phenomena based on electrical impedance tomography, and EIT data can be reconstructed by using the extracted components.

In an embodiment, respective components caused by changes of air in upper airway, changes in blood flow in the carotid, movement of the neck according to respiration, movement of the tongue, changes in air inside the lungs, or changes in thoracic blood flow are extracted from the EIT measurement data, and it is possible to restore an image based on a component caused by a specific physiological phenomenon. In addition, the quality of the restored image can be improved by flexibly setting current or voltage measurement ranges in consideration of the size and shape of the area to be imaged and increasing the number of voltages that can be distinguished from noise.

That is, according to the present inventive concept, as shown in FIG. 4, desired blood flow impedance data can be extracted by processing impedance data obtained from electrodes attached to the chest area. And the extracted blood flow impedance data is restored to an image as shown in FIG. 2, blood flow change information can be extracted from the restored blood flow image data.

In addition, as shown in FIG. 5, according to the present inventive concept, desired blood flow impedance data can be extracted by processing impedance data obtained from electrodes attached around to the wrist. Then, motion noise is removed from the extracted blood flow impedance data, and an image is restored as shown in FIG. 6, and blood flow change information can be extracted from the restored blood flow image data.

In addition, as shown in FIG. 7, according to the present inventive concept, desired blood flow impedance data can be extracted by processing the impedance data obtained from the electrodes attached around the neck. And an image is restored from the extracted blood flow impedance data as shown in FIG. 8, and blood flow change information can be extracted from the restored blood flow image data.

In addition, according to the present inventive concept, the blood flow image can be restored by processing the impedance data obtained by installing electrodes on a body part of arms, legs, etc. where blood vessels are located, as shown in FIG. 3.

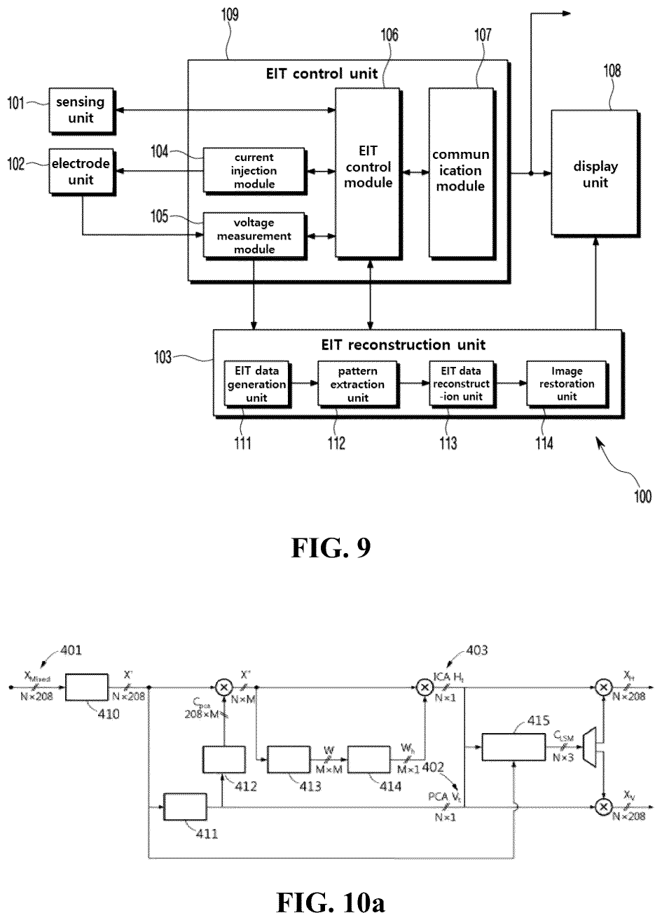

FIG. 9 is a control configuration diagram of a monitoring system according to an embodiment of the present inventive concept, shows the control configuration diagram for restoring a blood flow image using an impedance data selectively measured from blood vessels in various parts of the human body, as shown in FIG. 3. In addition, FIG. 9 can be used to restore a blood flow image and an airflow image by processing the impedance data measured in the chest area.

That is, the monitoring system 100 of the present inventive concept makes a pulmonary ventilation impedance image, a pulmonary perfusion impedance image, and a blood flow impedance image based on a pulmonary ventilation impedance data, a pulmonary perfusion impedance data, and a blood flow impedance data. And the monitoring system 100 comprises a display unit 108 configured to display the pulmonary ventilation impedance image, the pulmonary perfusion impedance image, the blood flow impedance image, and various images of the sensed biosignals, waveform signals, characters, measured values made of Arabic numerals, etc. as shown in FIG. 1. The display unit 108 can be configured integrally with the monitoring system 100 or configured to be separated from the monitoring system thereby capable of receiving signal from the monitoring system through a wired/wireless signal line and displaying the signal.

The monitoring system 100 of the present inventive concept comprises an electrode unit 102 attachable to each part of the human body as shown in FIG. 3. The electrode unit 102 is configured to be formed with a plurality of electrodes for current injection and voltage sensing and attached to a specific body part of a subject to be measured. Each of the plurality of electrodes can be at least one of a simple electrode or a composite electrode, and can be an EIT electrode for measuring impedance data attached to a corresponding part to be measured of a subject.

The EIT electrode is arranged on one surface of a base plate made of a flexible material and can be attached to a specific body part of the subject. The EIT electrode is used to inject a current with a safe strength (satisfied with IEC60601-1 standard), for example, a current of 1 mArms or less at a frequency of 10 kHz, to the subject and measure the induced voltage. The current-voltage data measured through the EIT electrode can be used to sense physiological changes inside the human body to which the electrode is attached through an imaging algorithm.

That is, the electrode unit 102 is configured to measure and receive impedance data from a subject.

The monitoring system 100 of the present inventive concept comprises a sensing unit 101 comprising various types of sensors for sensing biosignals of a human body. The sensing unit 101 can sense biosignals in contact with or a non-contact manner with a measurement target part of the human body. As an example, the sensing unit can comprise a plurality of sensors (i.e., fiber-based sensors) configured to sense biosignals of a subject. The plurality of sensors can be attached to different human body parts of the subject.

The sensing unit 101 can comprise at least one of a blood oxygen saturation measuring sensor that measures a blood oxygen saturation (SpO₂) signal of arterial blood according to the measurement target parts of the subject, a sound detecting sensor that detects sound according to biological activity of the subject, a posture measuring sensor that detects movement of the subject, an electrocardiogram measuring sensor that measures the electrocardiogram according to the measurement target part of the subject.

The blood oxygen saturation measuring sensor is configured to measure signal related to photoplethysmography (PPG) of a human body of a subject where light is reflected or transmitted, and to measure the blood oxygen saturation based on the measured photoplethysmography signal. The sound detecting sensor can detect at least one sound of breathing, snoring, crying, and sleep talk, and according to an embodiment, the sound detecting sensor is configured to be attached to a measurement target part of a subject during sleep or be in a non-contact form presented within a certain distance from the subject.

In addition, the posture measuring sensor is configured to be formed with at least one of a gyro sensor and an acceleration sensor, be attached to the measurement target part of the subject and measure the posture, ballistocardiogram, and seismocardiography according to movement.

The electrocardiogram measuring sensor is configured to be in contact with a measurement target part of the subject to measure an electrocardiogram (ECG). Wherein, the electrocardiogram (ECG) is a waveform composed of a sum of vectors for action potentials generated by a special excitatory & conductive system of a heart.

That is, the vector sum signal of the action potentials generated at each of components of the heart, such as SA (sinoatrial) node, AV (atrioventricular) node, His bundle, His bundle branch, purkinje fibers, etc. can refer to a signal measured from electrodes attached outside the body.

According to another embodiment, the sensing unit 101 can measure at least more than one of seismocardiography (SCG) and ballistocardiogram (BCG) of the subject.

And the sensing unit 101 is also capable of measuring the pressure data of air provided to the subject through a ventilator during a mechanical ventilation treatment process. Accordingly, the sensing unit 101 can measure respiration parameters related to respiration of the subject.

The monitoring system 100 of the present inventive concept comprises an EIT control unit 109.

The EIT control unit 109 is configured to selectively supply current to at least one or more electrode pairs selected from the plurality of electrodes, control to measure a voltage through the unselected electrodes, and be able to control to transmit the sensed signal, pulmonary ventilation impedance data, pulmonary perfusion impedance data, and blood flow impedance data.

The EIT control unit 109 comprises a current injection module 104. The current injection module 104 is configured to inject currents having a plurality of frequency ranges through at least one electrode pair selected among a plurality of electrodes attached to a specific part of the subject. The current injection module 104 is also configured to select frequency of the selected electrode pair, generates a voltage signal according to the selected frequency and converts the voltage into a current, and can inject the converted current into a specific part of the subject through the selected electrode pairs.

In another embodiment, the current injection module 104 is configured to convert a voltage signal into two currents having different phases, calibrate the two currents so that they are equal in amplitude and frequency, and inject two calibrated currents into the chest of the subject through the selected electrode pair.

The EIT control unit 109 comprises a voltage measurement module 105. The voltage measuring module 105 is configured to measure an induced voltage according to a current injected from the electrodes unselected among the plurality of electrodes. The voltage measurement module 105 is configured to remove noise included in the induced voltage based on the slope of the measured voltage, and when the slope of the detected voltage exceeds a preset threshold value, replace a voltage in a section exceeding the threshold value with a preset voltage value.

Accordingly, the EIT control unit 109 is configured to measure a plurality of electrical properties of the subject over time through the plurality of electrodes attached to the subject by using the current injection module 104 and the voltage measurement module 105. As an example, the EIT control unit 109 is configured to determine a supply electrode pair among a plurality of electrodes based on the circumference length of the measurement part and supply a current or a voltage to the supply electrode pair. In addition, the current or voltage induced from the current or voltage can be measured through the measuring electrode pair among the remaining electrodes except for the supply electrode pair among the plurality of electrodes. In addition, a plurality of impedance data can be measured in the voltage measurement range calculated by subtracting the measurement minimum value from the maximum measurement value.

The EIT control unit 109 is configured to change a method of injecting current or voltage in consideration of the size and shape of a region to be scanned as image and flexibly set a voltage or current measurement range accordingly. As an example, 208 time-series data can be generated by measuring about 208 impedance data by changing combinations of a supply electrode pair for injecting current or voltage from 16 electrodes and a measurement electrode pair for measuring voltage or current.

The EIT control unit 109 comprises an EIT control module 106. The EIT control module 106 is configured to control the selection of at least one or more electrode pairs in the plurality of electrodes, control the selection of unselected electrodes, and control sensing of the sensing unit 101 attached to the measurement target parts of the subject.

In addition, the EIT control module 106 is configured to control the EIT measurement by synchronizing to a specific time of the signal waveform measured by the sensing unit 101. For example, the EIT control unit 109 can be configured to control the current injection module 104 to measure impedance data for a specific part of the subject.

In addition, the EIT control module 106 can be configured to control the voltage measurement module 105 to measure the impedance data for the vertical and horizontal directions for a specific part of the subject and control the EIT reconstruction unit 103 that separates and reconstructs pulmonary ventilation impedance data, pulmonary perfusion impedance data, and blood flow impedance data from the measured impedance data and performs necessary image restoration.

And the EIT control module 106 can be configured to control the communication module 107.

The communication module 107 is configured to be included in the EIT control unit 109. The communication module 107 is configured to transmit pulmonary ventilation impedance data, pulmonary perfusion impedance data, blood flow impedance data, and other biological signals which are signal-processed in the monitoring system 100 of the present inventive concept, to the outside through wired/wireless communication.

In addition, the EIT reconstruction unit 103 can be configured to be included in the EIT control unit 109 as a single module (or unit) or configured to be a separate form. In the illustrated embodiment, these two units are separately configured. The EIT reconstruction unit 103 can be configured to separate the pulmonary ventilation impedance data, the pulmonary perfusion impedance data, and the blood flow impedance data from the sensed impedance data. In addition, the EIT reconstruction unit 103 can be configured to reconstruct the separated EIT data and restore an impedance image for the corresponding data.

The EIT reconstruction unit 103 comprises an EIT data generation unit 111 that generates EIT data based on changes of a plurality of electrical properties measured by the voltage measurement module 105. The generated EIT data can be expressed as a measured EIT data.

The EIT data generation unit 111 is configured to generate an EIT data according to a voltage measurement range. The EIT data generation unit 111 is configured to generate an EIT data between maximum and minimum measurement values of voltages. The generated EIT data can include changes in a plurality of electrical properties, noise, and motion noise, etc. As an example, the EIT data can be affected by impedance changes due to a stenosis of upper airway, a respiratory movement, a blood flow in the carotid artery, and an irregular movement of the mandible and tongue.

The EIT reconstruction unit 103 comprises a pattern extraction unit 112 that determines at least one or more pattern data from the generated EIT data by using the signal-to-noise ratio of the generated EIT data. The EIT data can include a plurality of different signal-to-noise ratios based on a plurality of electrical properties. That is, the pattern extraction unit 112 can determine pattern data corresponding to changes of 16 electrical properties having a good signal-to-noise ratio among changes of 208 electrical properties constituting the EIT data. The pattern data can be referred to as frequency pattern data related to a scale change of electrical properties.

The pattern extraction unit 112 can be configured to extract pattern data corresponding to a specific component generated from a physiological phenomenon of a subject among at least one or more pattern data. The specific component can include at least one of a change in air inside the lungs or airway of the subject, a change in blood flow inside the body, a change in a component inside the body, and a change in movement of a part of the body.

The pattern extraction unit 112 is configured to extract any one of energy or frequency of EIT data using any one of signal-to-noise ratio in the EIT data, principal component analysis (PCA), or independent component analysis (ICA). The pattern extraction unit 112 is configured to extract a specific pattern data related to a specific component generated from a specific physiological phenomenon of the subject based on the frequency component according to the extracted energy or frequency.

Accordingly, the pattern extraction unit 112 is configured to extract only a component due to a specific physiological phenomenon from a composite signal that is affected by changes in electrical properties inside the human body according to a plurality of physiological phenomena. That is, components due to changes in air in upper airway, changes in blood flow in a carotid artery, movements of a neck according to respiration, movements of a tongue, changes in air inside lungs, or changes in thoracic blood flow can be respectively extracted from the EIT measurement data.

The EIT reconstruction unit 103 comprises an EIT data reconstruction unit 113 that reconstructs the EIT data into an EIT data (reconstructed) corresponding to a specific component by using the extracted pattern data. The EIT data reconstruction unit 113 is configured to reconstruct the EIT data into an EIT data (reconstructed) corresponding to a specific component by using a difference in a relative voltage change between the extracted specific pattern data and the EIT data. The EIT data reconstruction unit 113 is configured to rescale the EIT data by using the least-squares error method because the difference in the relative sizes of the pattern data measured for a predetermined time is mutually the same.

The EIT reconstruction unit 103 comprises an image restoration unit 114 that restores an image related to a specific component by using the reconstructed EIT data. When a specific component is a component due to a change in air inside the lungs or a change in blood flow in the chest, the image restoration unit 114 can be configured to separately restore each of images of a change in air in the lungs and an image of a change in blood flow in the chest, respectively.

The image restoration unit 114 can be configured to improve the quality of the restored image by increasing the number of voltages that are distinguished from noise by flexibly setting the voltage or current measurement range.

The monitoring system 100 according to the embodiment of the present inventive concept configured as described above can display the EIT image and data related to the hemodynamic diagnostic parameters through the following processes.

In the following description, a process of restoring a blood flow image by processing impedance data detected through electrodes attached to a neck of a subject will be described as an example.

As shown in FIG. 7a, the EIT control unit 109 is configured to determine a supply electrode pair among a plurality of electrodes based on perimeter length of a measurement part, and as shown in FIG. 7b, supply current or voltage to the supply electrode pair. In addition, the EIT control unit 109 is also configured to measure the current or voltage induced from the current or voltage through the measuring electrode pair among the remaining electrodes except for the supply electrode pair among the plurality of electrodes. In addition, the EIT control unit 109 is configured to measure a plurality of impedance data in the voltage measurement range calculated by subtracting the measurement minimum value from the measurement maximum value.

That is, a plurality of electrical properties including changes in components such as breathing related motion noise, blood flow, upper airway occlusion, etc. are measured through the electrode unit 102 attached to a neck of a subject. Noise according to the movement of the subject can be added to the plurality of measured electrical properties.

The EIT data generation unit 111 is configured to generate EIT data based on changes of a plurality of measured electrical properties. The generated EIT data can have been affected by impedance changes due to stenosis of upper airway, respiratory movement, blood flow in carotid artery, and irregular movements of jaw and tongue.

The pattern extraction unit 112 is configured to select 16 voltage channels having highest signal-to-noise ratio (SNR) among 208 time-series voltage channels as input channels of the ICA algorithm. The determined pattern data can correspond to ICA components.

The pattern extraction unit 112 is configured to remove noise pattern data among 16 ICA components. In addition, respiration motion and blood flow components can be identified through spectrum analysis of an independent source signal S. If Fast Fourier Transform is applied to all independent components of the independent source signal, components with fundamental frequencies corresponding to respiration rate and heart rate can be identified as respiration motion and blood flow components, respectively. The corrected source signal U can be calculated using the following Equation 1.

U=W⁻¹S (Equation 1)

Wherein, W⁻¹ represents a corrected mixing matrix, and S represents an independent source signal. W⁻¹ is calculated by substituting 0 column for the column corresponding to the identified components of respiratory movement and blood flow.

As an example, the pattern extraction unit 112 is configured to extract pattern data corresponding to a specific component corresponding to upper airway signal, and a filtered result can be shown as FIG. 8d. According to FIG. 8d, the graph 320 can correspond to a specific component, and the graph 321 can correspond to upper airway signal that has passed through a low-pass filter.

At this time, 208 voltage data corresponding to upper airway stenosis can be restored to an appropriate amplitude. In addition, a low-pass filter can also be used to reduce residual noise of the recovered voltage data without distorting the pattern of upper airway stenosis (constriction). Next, the EIT data reconstruction unit 103 can be configured to reconstruct an EIT data including 208 electrical property changes corresponding to a specific component by using the extracted pattern data based on Equation 2 as below.

Vⱼ=aⱼU_{UA}+bⱼ (Equation 2)

According to Equation 2, V; represents voltage of the j-th channel, and U represents a corrected source signal. aⱼ and bⱼ are constants corresponding to difference values between 208 voltage data, and matrix data C transformed to calculate Vⱼ can correspond to Equation 3.

$$C = {[ \begin{matrix}{a}_{1} & … & {a}_{208} \\ {b}_{1} & … & {b}_{208}\end{matrix} ]}^{T} \qquad ( Equation 3 )$$

According to Equation 3, C is a matrix of 208 voltage data correction constants. Transpose (T) of a matrix is used to alter the expression. The voltage correction constant matrix C can be reconstructed based on Equation 4 by using the original signal X of the voltage and the corrected source signal U.

C=XU_{UA}^{T}(U_{UA}U_{UA}^{T})⁻¹ (Equation 4)

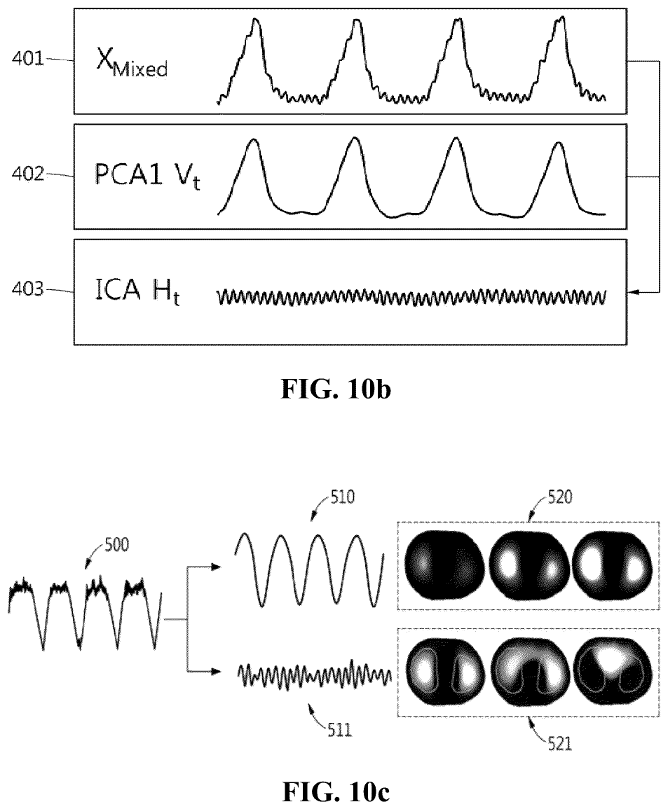

FIG. 10a shows a diagram illustrating a configuration for reconstructing extracted pattern data according to an embodiment of the present inventive concept.

That is, the EIT data reconstruction unit 113 is configured to remove the background noise in a BAR processing unit 410 when a mixed signal 401 is applied. A PCA processing unit 411 extracts PCA pattern data 402 corresponding to the voltage principal component of the signal from which the background noise is removed. The PCA pattern data 402 is extracted and outputted as respiratory component related data.

The L-curve searching unit 412 is configured to extract L-curve data from the PCA pattern data 402 and search for dimension-reduced voltage component data used in the ICA processing unit 413. Then, the ICA selection unit 414 is configured to select and output ICA pattern data 403 corresponding to a specific component among the ICA components.

The source comparison unit 415 is configured to confirm homogeneity of the PCA pattern data 402 and the ICA pattern data 403, and the EIT data reconstruction unit 113 is configured to reconstruct the EIT data using the PCA pattern data 402 and the ICA pattern data 403, respectively.

FIG. 10b shows, as an example, frequency patterns of mixed signal 401, PCA pattern data 402, and ICA pattern data 403, the mixed signal 401 includes the PCA pattern data 402 and the ICA pattern data 403.

As such, when the pattern data of a specific component is extracted by the pattern extraction unit 112, the EIT data reconstruction unit 113 is configured to generate by reconstructing an EIT data 500 corresponding to a mixed signal, as shown in FIG. 10c. That is, pattern data corresponding to a respiratory component 510 can be extracted, and pattern data corresponding to a blood flow 511 can be extracted.

As described above, the present inventive concept makes it possible to separate components caused by air change or blood flow change from EIT measurement data by using the measured impedance data, and to separately restore an air change image 520 and a blood flow change image 521 by using the separated EIT data.

In an embodiment of the present inventive concept, the image restoration unit 114 is configured to restore image data resulting from a blood flow change measured in a neck area by using a blood flow.

At least one according to the degree and shape of hemodynamic changes in a heart and blood vessels, hemodynamic diagnostic parameters, etc., is quantified based on a blood flow image including restored blood flow change information of upper airway.

To this end, the EIT control module 106 is configured to obtain at least 100 or more restored blood flow images per second, set the blood vessel region as a region of interest (ROI) in the blood flow image, and extract a change in pixel values in the ROI as a blood flow change signal.

As an example, 100 or more frames per second are extracted from the restored blood flow images, and the extracted multiple frame images are temporarily stored in a memory. Then, the characteristics of each frame image are analyzed, and a relationship between pixels adjacent to each other in each frame image is obtained. Then, each frame image is divided into a plurality of color blocks according to the characteristics of each frame image, and identification data is set in each color block and stored in the memory. In this way, a plurality of pixel values are stored in the memory, and identification data related to color block belonging to each pixel value is stored. A change amount of each color block is formed by comparing the change amount of each pixel value in each color block between previous and subsequent frames, and the change amount of each color block is calculated as an average value of the change amount in previous and subsequent frames for a plurality of pixel values in each color block.

In this way, when pixel values in ROI are calculated to generate a time-varying signal, the signal is extracted as a blood flow change signal based on changes in the pixel values in the ROI. Therefore, the change in the magnitude of the blood flow change signal is calculated in connection with at least any one or more measurement data among the average deviation, average dispersion, average phase delay, and average absolute impedance value according to the change in the impedance data calculated based on the blood flow image, and a stroke volume is calculated based on the blood flow change signal.

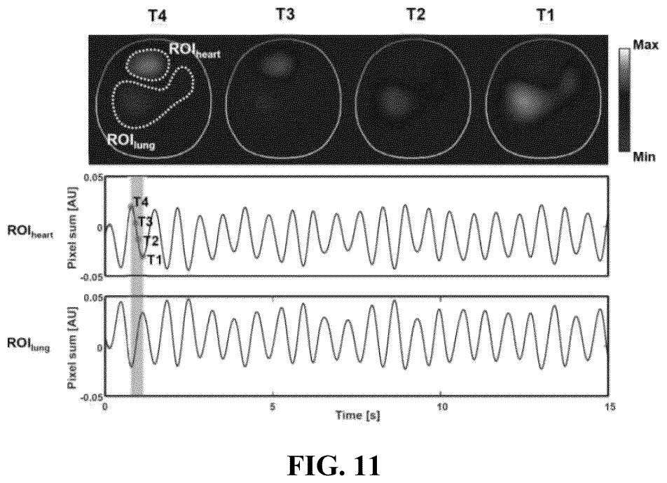

FIG. 11 shows a graph showing the sum of pixel values based on a blood flow change signal in the region of interests (ROIs) set in the heart and lungs. That is, it can be shown that region of interest of the heart (ROIₕₑₐᵣₜ) has the highest blood flow change signal at time T4, and region of interest (ROI_{lung}) has the highest blood flow change signal at time T1. This blood flow change signal as shown above is expressed as a sum of pixel values. This blood flow change signal as shown above can be defined as a stroke volume value.

In another embodiment, the EIT control module 106 is configured to apply to calculate the absolute value of a stroke volume by using personal information such as age, gender (sex), weight, height, etc. of a subject together with EIT measurement data. To this end, the EIT control module 106 is configured to set weights according to the age, gender (sex), weight, height, etc. of the subject based on the experimental values, and store the weights in a memory or the like. In addition, it is also possible to calculate the stroke volume by applying corresponding weights.

And it is possible to calculate a cardiac output by using stroke volume and heart rate as follows.

Wherein, the heart rate is a value measured through a sensor included in the sensing unit 101.

Cardiac Output=Stroke Volume×Heart Rate

In addition, it is possible to calculate a peripheral vascular resistance by using the calculated cardiac output and measured blood pressure as follows. Wherein, the blood pressure is a value measured through a sensor included in the sensing unit 101.

Peripheral Vascular Resistance=Blood Pressure/Cardiac Output

As described above, it is possible to scan a blood flow image of carotid artery by using impedance value measured through electrodes attached to a neck, and it is possible to calculate hemodynamic diagnostic parameters such as stroke volume, cardiac output, and peripheral vascular resistance from the blood flow image. The hemodynamic diagnostic parameters calculated as described above can be displayed through the display unit 108 together with the blood flow image of the corresponding part.

In addition, the EIT control module 106 is configured to transmit the measured various hemodynamic diagnostic parameters to outside by wire or wirelessly through the communication module 107. In addition, when the measured value appears higher than a value preset to determine a dangerous state of the subject, it is also possible to output a warning message or a warning sound to the display unit 108.

FIG. 8a to FIG. 8c show a stroke volume graph and an electrocardiogram graph obtained in proportion to a blood flow impedance image measured in a neck. And, as shown in FIG. 1, it is possible to display stroke volume, cardiac output, and peripheral vascular resistance value with Arabic numerals and characters (or letters).

Therefore, according to the present inventive concept, it becomes possible to perform selectively scanning electrical impedance tomography for blood vessels in any part of a chest, neck, arms, legs, etc., and monitoring hemodynamic diagnostic parameters comprising a stroke volume, a cardiac output, and a peripheral vascular resistance. In particular case of critically ill patients where it can be difficult to attach electrodes to the chest and continue to perform scanning electrical impedance tomography, it is possible according to the present inventive concept to perform monitoring hemodynamic diagnostic parameters from EIT images scanned from other blood vessel parts. In addition, in the case of a critically ill patient, the present inventive concept enables real-time monitoring of hemodynamic diagnostic parameters that change in real-time during a treatment process such as drug injection. Therefore, it becomes possible for medical staff to check change status of the patient in real-time, and to appropriately support the process of treatment and diagnosis prediction for the patient.

On the other hand, the monitoring system 100 of the present inventive concept can be configured to constitute the following embodiments.

As can be seen from the state shown in FIG. 5, it is also possible to use a data collection unit using a computer. That is, electrodes are attached to circumference of a specific part of the human body for scanning EIT image, and current is injected by using selected electrodes. Then, the voltage is measured by using the remaining unselected electrodes. Accordingly, according to the present inventive concept, it is possible to comprise a data collection unit for collecting the measured voltage signals.

In addition, according to the present inventive concept, it is possible to restore airflow image and blood flow image, respectively, by using a data processing device composed of various software and hardware provided in a computer by using the EIT measurement data collected by the data collection unit. The data processing device can also extract hemodynamic diagnostic parameters or respiratory mechanics diagnostic parameters which are state variables for each region of the lungs, from each of the airflow image and the blood flow image. In this case, the data processing device and the data collection unit are configured to comprise the components shown in FIG. 9.

Hereinafter, processes of measuring state parameters for each area of the lungs in real-time by using the ventilation EIT image and displaying the measured state parameters as images will be described.

The monitoring system 100 according to an embodiment of the present inventive concept shown in FIG. 9 is configured to output and display a temporal change in the relative sizes of the collapsed region and hyperinflated region of the lungs during the mechanical ventilation treatment process. Therefore, in the same manner as in the process of sensing hemodynamic diagnostic parameters from the blood flow EIT image described above, a ventilation EIT image is constructed from the correlation of various components of FIG. 9, a process of sensing respiratory dynamics diagnostic parameters is performed based on the ventilation EIT image.

It is impossible for conventional CT or X-rays to perform real-time or continuous monitoring.

However, the monitoring system 100 according to an embodiment of the present inventive concept is configured to measure impedance data through electrodes attached around a chest, and check states of pulmonary compliance, ventilation delay, etc. in real-time and continuously from the measured impedance data. In particular, according to the present inventive concept, it is possible to perform checking simultaneously with the treatment process according to mechanical ventilation.

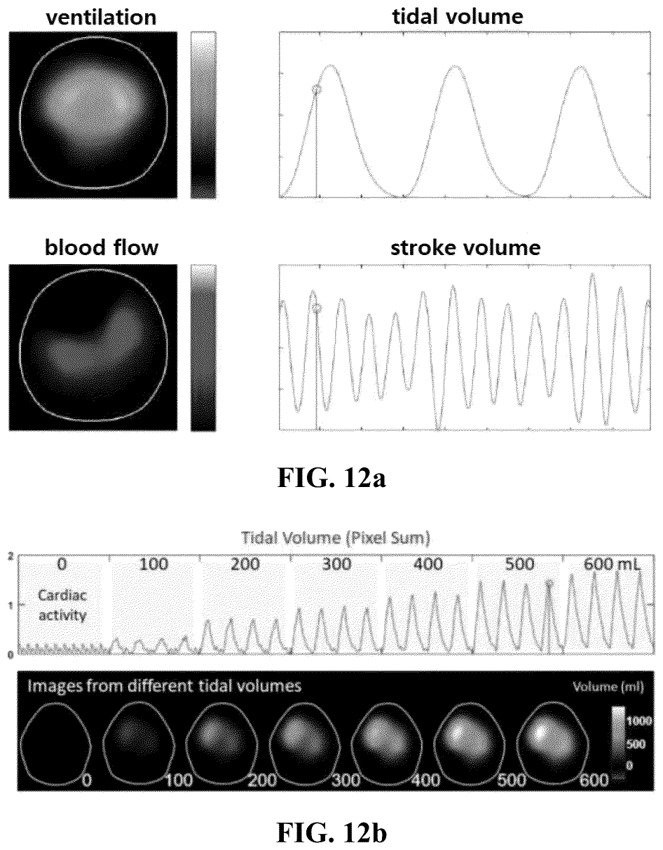

As shown in FIG. 12a, the monitoring system of the present inventive concept is configured to implement a respiration image and a tidal volume graph interlocked therewith the respiration image, and a blood flow image and a stroke volume graph interlocked therewith the blood flow image. That is, it is possible to implement a blood flow EIT image by extracting only a blood flow component from impedance data measured from the electrodes, or a ventilation EIT image by extracting only a respiration component from the measured impedance data.

As a stroke volume graph is implemented by extracting blood flow change signals from changes in pixel values in the blood flow EIT image shown in FIG. 11, a tidal volume graph can be implemented by extracting respiration change signals from changes in pixel values in the respiration EIT image in FIG. 12b.

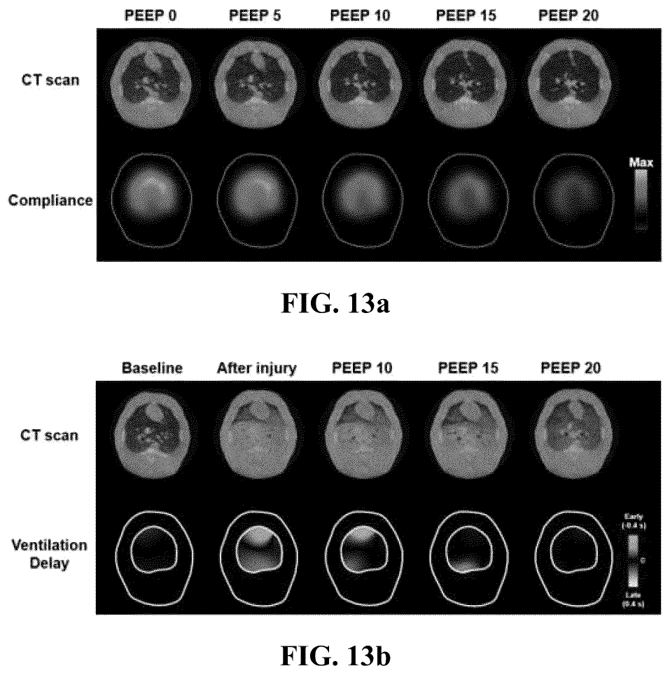

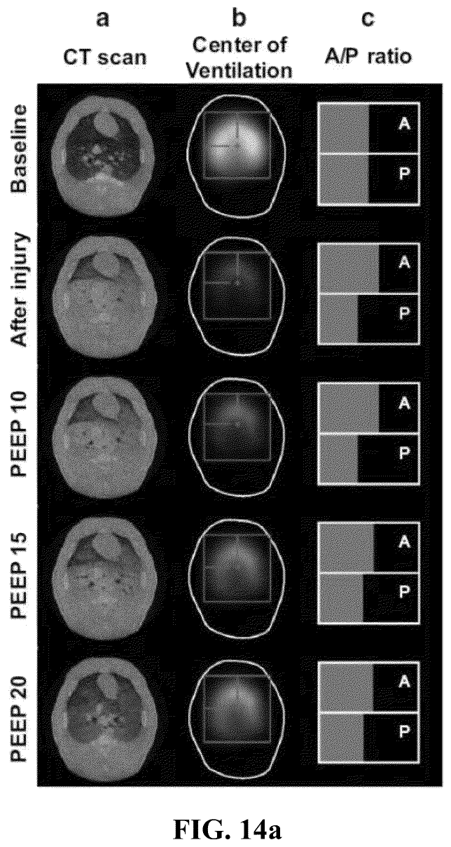

FIG. 13a shows an image of pulmonary compliance obtained from an animal undergoing mechanical ventilation through an animal experiment. The image being shown shows that pulmonary compliance decreases as PEEP increases. And the CT scan image is shown for comparison with the image scanned by the monitoring system of the present inventive concept.

That is, the CT scan image shows increase in lung volume with increase in PEEP.

In addition, FIG. 13b shows a ventilation delay image obtained through an animal experiment.

It can be easily seen that the ventilation delay image is significantly different in normal and collapsed cases in the image shown. It can be seen that pixel values in the ventilation delay image are reduced after injury as in the image shown. And when PEEP increases, it can be seen that the difference in pixel values in the ventilation delay image is reduced, and the collapsed area of the lung is shrinking. In addition, the CT image is shown for comparison with the scanned image of the monitoring system of the present inventive concept, and it can be seen that the collapsed area in the CT scan image is also reduced according to increase in PEEP.

That is, as shown in FIG. 13a, it is possible to see the pulmonary compliance image as seen in a conventional CT image through the EIT reconstruction image according to an embodiment of the present inventive concept. In addition, as shown in FIG. 13b, it is possible to see the ventilation delay image as seen in a conventional CT image through the EIT reconstruction image according to an embodiment of the present inventive concept.

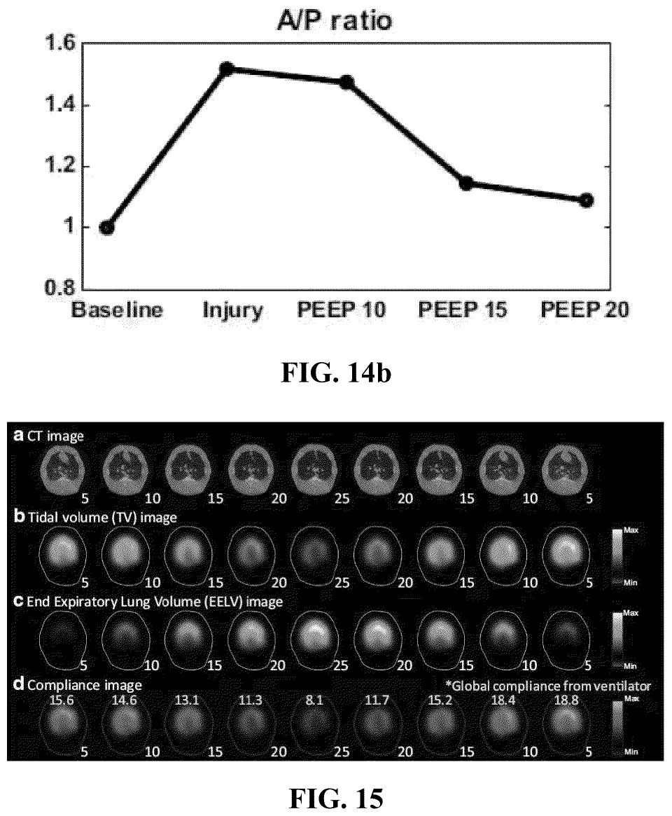

And FIG. 14a shows state of the image that changes according to the increase of PEEP from the central pixel of the ventilation image. The CT scan image (a) is a component added to be compared with the respiratory image of the present inventive concept. And the change of ventilation along the vertical direction from the central pixel of the ventilation EIT image (b) according to the amount of change in PEEP is expressed as an A/P (anterior-to-posterior ventilation) ratio value (c), and the amount of the change is shown as a graph in FIG. 14b. That is, according to the present inventive concept, it can be seen that the A/P ratio value is approaching normal value as the positive end-expiratory pressure increases, and the ventilation image seen by the EIT image is closer to the normal value.

As described above, the present inventive concept uses the monitoring system 100 to measure state parameters for each region of the lungs in real-time and display the measured state parameters as images. And detecting collapse and hyperinflation inside the lungs is performed by analyzing the volume and pressure data of air applied to a patient by a ventilator used in mechanical ventilation process together with the EIT ventilation image that provides information on the change in the amount of air inside the lungs.

Therefore, the electrode unit 102 should be configured to obtain respiratory impedance data.

As an example, the impedance data including respiration and blood flow components can be obtained around a chest. Therefore, it is preferable that the electrode unit 102 is attached around to a chest of a subject for the purpose of detecting collapse and hyperinflation inside the lungs.

Impedance data obtained based on values detected from the electrode unit 102 is reconstructed to respiratory impedance data required through the EIT reconstruction unit 103, and a respiration image is restored in the image restoration unit 114. The EIT control module 106 is configured to calculate pulmonary compliance and ventilation delay that can express collapse and hyperinflation of the lungs by using the reconstructed respiration image.

The EIT control module 106 is configured to acquire at least 25 or more restored respiratory images per second, set a certain part as a region of interest (ROI) in the respiratory images, and extract changes in pixel values in the ROI as a respiration change signal (or an airflow change signal). It can be variably set the number (number of frames) to obtain the restored respiration image per second in the EIT control module 106.

As an example, FIG. 12a shows a reconstructed respiration image that varies according to a respiration volume as an EIT image according to a cardiopulmonary function detected during an animal experiment. And a tidal volume realized by calculating the sum of pixel values from the restored respiration image is expressed as a graph. Accordingly, FIG. 12b as illustrated shows that one respiration image (TV) is generated for each respiration cycle, and respiratory images (TV) that are sequentially shown shows changes in ventilation according to several respiration cycles.

Therefore, the EIT control module 106 is configured to extract more than 25 frames per second of the restored respiration image, and temporarily store the extracted multiple frame images in memory. And then the EIT control module 106 is configured to analyze the characteristics of each frame image and obtain a relationship between pixels adjacent to each other in each frame image. Then, each frame image is divided into a plurality of color blocks according to the characteristics of each frame image, and identification data is set in each color block and stored in the memory. In this way, values of a plurality of pixels are stored in the memory, and identification data related to the belonging color block is stored for each pixel value. The EIT control module 106 is configured to form a change amount of each color block by comparing the amount of change between preceding and subsequent frames for each pixel value in each color block and calculate the amount of change in each color block as an average value of the amount of change between the preceding and subsequent frames for a plurality of pixel values in each color block.

As the above, through generating a signal that changes with time by calculating pixel values in the region of interest, the signal is extracted as a respiration change signal based on changes in pixel values in the region of interest. Therefore, the tidal volume is calculated based on the respiratory change signal, wherein the magnitude change of the respiratory change signal is calculated in connection with at least any one or more measurement data of the average deviation, average dispersion, average phase delay, and average absolute impedance value according to the change in impedance data calculated based on the respiratory impedance image.

At this time, weights for personal information such as age, gender (sex), weight, and height of the subject are set in advance and are given as the corresponding weights, and thus the weights can be used together with EIT measurement data and applied to calculate the absolute value of a tidal volume.

In addition, desired pulmonary compliance data can be obtained by computing a ventilation volume (ΔV) extracted from each pixel and the pressure (ΔP) applied by the ventilator. That is, the pulmonary compliance data (C) is a change in volume that occurs according to a change in unit pressure and is the value of pulmonary compliance in one respiration at each pixel. And it is possible to generate an image of pulmonary compliance in one respiration by using the pulmonary compliance data obtained from each pixel.

Pulmonary compliance (C)=ventilation volume (ΔV)/pressure applied by the ventilator (ΔP) FIG. 15 shows an image changed when different PEEP values are sequentially applied in the order of 5, 10, 15, 20, 25, 20, 15, 10, and the like. And the CT image (a) is shown in order to compare the pulmonary compliance state with the pulmonary compliance image implemented in the present inventive concept. It can be shown that the lungs are gradually expanding as PEEP value increases in CT image (a).

And like the CT image (a) which is changed according to the change in PEEP, it is possible to be seen as comparing the respiratory image change state (b) and the pulmonary compliance image (d) change according to the change in respiration volume reconstructed through the configuration of the present inventive concept. And it can be shown that the image (c) according to the end-expiratory lung volume change (EELV) increases as the PEEP value increases. That is, it can be shown that the EIT image reconstructed in the present inventive concept can be used to quantitatively measure the air distribution in the lungs in real-time.

As described above, it is possible to see the real-time changing state of the subject from the tidal volume image obtained from the respiration image, the image according to the change in the end expiratory lung volume, and the pulmonary compliance image. The Arabic numerals at the bottom of each image are the values of PEEP. Therefore, according to the present inventive concept, it is possible to see in real-time a pulmonary compliance image that changes according to a change in the PEEP value from the PEEP treatment process.

As such, it can be shown that the pulmonary compliance data decreases with the increase of PEEP in the pulmonary compliance image. However, in the course of mechanical ventilation treatment, there is a concern in occurring of ventilation delay in pixels included in other regions of the lung due to an increase in PEEP. Therefore, the monitoring system of the present inventive concept is characterized by calculating the ventilation delay during one respiration in each pixel for each part (region) of the lung.

Regional ventilation delay (RVD) is the ventilation delay for each pixel, and the calculation of ventilation delay can be seen as below, by calculating the time (Δt^{40%}) taken to reach the volume corresponding to 40% of the maximum volume from the start of inspiration in the corresponding pixel, with respect to the time from the start of inspiration to the end of inspiration (tₘₐₓ−tₘᵢₙ). That is, the ventilation delay is calculated at each pixel by using the time when the respiration impedance arrives at 40% of the maximum value. Wherein, the maximum lung volume is measured by using the volume of air applied to a patient by a ventilator used in the mechanical ventilation process. And the 40% point in time (Δt^{40%}) is a value set based on the experimental value, and is a value set as the most optimal time point because the present inventive concept requires fast signal processing in the treatment process that requires real-time display and monitoring.

Regional ventilation delay (RVD)={Δt^{40%}/(tₘₐₓ−tₘᵢₙ)}×100%

And it is possible to generate a ventilation delay image as shown in FIG. 13b using the calculated ventilation delay data in each pixel.

Therefore, the present inventive concept is configured to calculate pulmonary compliance data and ventilation delay data from the ventilation EIT image, generate an image of pulmonary compliance by using the obtained pulmonary compliance data, and generate a ventilation delay image by using the ventilation delay data.

In general, pulmonary compliance is reduced in the regions of collapse and hyperinflation of the lungs. Accordingly, it becomes possible to diagnose the collapsed and hyperinflated regions of the lungs from the pulmonary compliance image generated according to the present inventive concept.

Ventilation delay is also increased in the collapsed region of the lungs. Therefore, it becomes possible to diagnose the collapse and hyperinflation regions of the lungs from the pulmonary compliance image, and again, it becomes possible to distinguish the collapsed region of lungs from the hyperinflated region of lungs from the ventilation delay image generated according to the present inventive concept. And since the remaining regions except for the collapsed and overinflated regions of the lungs become the normal regions, it is possible to calculate as a ratio for the collapse regions of the lungs, the overinflated regions of the lungs, and the normal regions of the lungs, and monitor the ratio in real-time.

As described above, according to the present inventive concept, it is possible to selectively scan electrical impedance tomography of blood vessels in any part of the chest, neck, arms, legs, etc., and to monitor hemodynamic diagnostic parameters including a stroke volume, a cardiac output, a peripheral vascular resistance, etc. In particular, according to the present inventive concept, in the case of a critically ill patient, it becomes possible to monitor in real-time hemodynamic diagnostic parameters that change in real-time during a treatment process such as drug injection. Therefore, it becomes possible for medical staffs to check the change status of the patient in real-time, and to appropriately support the processes of treatment and diagnosis prediction for the patient.

In addition, according to the present inventive concept, it is possible to monitor the state parameters for each region of the lungs in real-time by using the same monitoring device. In particular, it is possible to calculate pulmonary compliance data by using the respiration change signal in each pixel obtained from the ventilation EIT image of the lungs, and to generate the pulmonary compliance image from the calculated pulmonary compliance data. In addition, it is possible to calculate ventilation delay generated during one breath in each pixel, and to generate a ventilation delay image by using the ventilation delay data. It becomes possible to diagnose collapse and hyperinflation of the lungs by comparing the generated ventilation delay image with the lung compliance image, and to induce to inject appropriate end-tidal positive pressure to the subject.

The above detailed description should not be construed as restrictive in all respects and should be considered as exemplary. The scope of the present inventive concept should be determined by a reasonable interpretation of the appended claims, and all modifications within the equivalent scope of the present inventive concept are included within the scope of the present inventive concept.

1. A system for monitoring cardiopulmonary function using electrical impedance tomography, comprising:
an electrode unit configured to measure impedance data by attaching a plurality of electrodes to at least one region with blood vessels of chests, a neck, arms, legs, or wrists of a subject;
an image restoring unit configured to restore an EIT image by extracting blood flow impedance data from the measured impedance data; and
an EIT control module configured to set a region of interest in the restored EIT image, extract a blood flow change signal based on change amount of pixel values inside the region of interest, and calculate hemodynamic diagnostic parameters by using the extracted blood flow change signal.

2. The system of claim 1,
wherein the EIT control module is further configured to calculate a stroke volume by using the extracted blood flow change signal.

3. The system of claim 2,
wherein the EIT control module is further configured to a cardiac output by computing a heart rate measured from the subject on the calculated stroke volume.

4. The system of claim 2,
wherein the EIT control module is further configured to calculate a peripheral vascular impedance by computing the blood pressure measured from the subject on the cardiac output.

5. The system of claim 2,
wherein the EIT control module is further configured to calculates lung perfusion by extracting blood flow change in the lung region of the subject.

6. The system of claim 2,
wherein the EIT control module is further configured to preset weights according to gender, age, height, and weight of the subject and apply the preset weights to the stroke volume calculation.

7. The system of claim 1,
The system further comprising:
a display unit configured to display the EIT image restoring a blood flow change signal over time generated based on a signal detected in real-time through the electrode unit, a graph of hemodynamic diagnostic parameters proportional to the EIT image, and numerical values.

8. A system for monitoring cardiopulmonary function using electrical impedance tomography, comprising:
an electrode unit configured to measure impedance data by attaching a plurality of electrodes to a chest of a subject for monitoring collapse and hyperinflation of lungs in real-time during the mechanical ventilation treatment process;
a sensing unit configured to measure pressure data of air applied to the subject in the mechanical ventilation treatment process;
an image restoring unit configured to restore an EIT image by extracting airflow impedance data from the measured impedance data; and
an EIT control module configured to obtain a plurality of airflow EIT images in order to extract an airflow change signal from the restored EIT image, extract the airflow change signal in each pixel based on the change in the pixel value from the obtained EIT images, and calculate respiratory dynamics diagnostic parameters by using the extracted airflow change signal.

9. The system of claim 8,
wherein the EIT control module is further configured to calculate a stroke volume by using the extracted airflow change signal.

10. The system of claim 9, further comprising:
a display unit configured to display lung compliance data as images, wherein the lung compliance data is changed in synchronization with time change,
wherein the EIT control module is further configured to calculate the lung compliance data in each pixel by computing a ventilation volume and air pressure data extracted from each pixel.

11. The system of claim 10,
wherein the EIT control module is further configured to calculate a ventilation delay data by computing time taken to reach a volume corresponding to 40% of maximum volume from start of inspiration in the corresponding pixel, with respect to the time taken from the start of inspiration to the end of inspiration, and
wherein the display unit is further configured to display the ventilation delay data as images, wherein the ventilation delay data is changed in synchronization with time change.

12. The system of claim 10,
wherein the EIT control module is further configured to determine an area as collapse and hyperinflation of the lungs, by combining results determined from lung compliance data and the ventilation delay data,
wherein the area in which the lung compliance data is reduced within each cycle of respiration is determined as the collapse area and the hyperinflation area of the lungs, and the area in which the ventilation delay data increases within each cycle of respiration is determined as the collapse area of the lungs.

13. The system of claim 12,
wherein the EIT control module is further configured to calculate results of lung compliance data and ventilation delay data according to changes where positive end-expiratory pressure (PEEP) increases and decreases,
the display unit is further configured to display the collapsed and overinflated areas of the lungs that are changed in synchronization with changes of positive end-expiratory pressure.

14. A method for monitoring cardiopulmonary function using electrical impedance tomography, comprising:
measuring impedance data by attaching a plurality of electrodes to a chest of a subject;
measuring air pressure data and air volume data applied to the subject during the mechanical ventilation treatment process;
restoring a blood flow EIT image and an airflow EIT image by extracting a blood flow impedance data and an airflow impedance data from the measured impedance data;
extracting a blood flow change signal based on a change amount of a pixel value inside a region of interest, by obtaining a plurality of EIT images for a predetermined time, and setting a blood vessel region as the region of interest in the obtained EIT image, in order to extract a blood flow change signal from the restored blood flow EIT image;
extracting an airflow change signal in each pixel based on a change in pixel value from the obtained EIT image by obtaining a plurality of airflow EIT images for a predetermined time, in order to extract an airflow change signal from the restored airflow EIT image; and
calculating hemodynamic diagnostic parameters by using the extracted blood flow change signal and respiratory dynamics diagnostic parameters by calculating the airflow change signal and air pressure data extracted from each pixel.

15. The method of claim 14,
the extracting of the blood flow change signal, comprises:
extracting the blood flow change signal from the blood flow impedance data obtained from a body part with a blood vessel of a chest, neck, arms, legs, and wrists of the subject.

16. The method of claim 14, further comprising:
displaying the EIT image restoring a blood flow change signal generated based on a signal detected in real-time from the electrodes, a graph of hemodynamic diagnostic parameters calculated from the EIT image, and a numerical value.

17. The method of claim 14,
wherein the extracting of the airflow change signal further comprising:
extracting the airflow change signal with respect to a tidal volume, a lung compliance data, ventilation delay data, by using the airflow impedance data and air pressure data obtained from the part where there is air flow due to respiration in the neck and chest of the subject.

18. The method of claim 17, further comprising:
displaying an EIT image restoring an airflow change signal generated based on a signal detected in real-time from the electrodes, a graph of respiratory dynamics diagnostic parameters calculated from the EIT image, and numerical values.
